(19) United States
(12) Patent Application Publication
(10) Pub. No.: US 2023/0210686 A1
(43) Pub. Date: Jul. 6, 2023

(54) Menstrual Disc

(71) Applicant: Diva International Inc., Kitchener (CA)

(72) Inventor: Carinne Saini, Kitchener (CA)

(21) Appl. No.: 18/067,173

(22) Filed: Dec. 16, 2022

Related U.S. Application Data
(60) Provisional application No. 63/297,088, filed on Jan. 6, 2022.

(51) Int. Cl. A61F 5/455

(52) U.S. Cl. CPC A61F 5/4553

(57) ABSTRACT

A menstrual disc is provided. The disc comprises a receptacle that has a posterior end, an anterior end and a perimeter, said receptacle comprising a wall having an inner surface and an outer surface, wherein the wall extends from an open top end to a closed bottom end, the inner surface of the wall defining a reservoir adapted to collect fluid, the wall of the reservoir extending from the bottom to the anterior end having a slope that is less than the slope of the wall from the bottom to the posterior end; and a rim formed at the top end of the receptacle which extends around the perimeter of the receptacle. The unique shape of the disc minimizes leakage during use and permits use during intercourse.

### FIELD OF THE INVENTION

The present invention generally relates to menstrual products such as menstrual cups and discs.

### BACKGROUND OF THE INVENTION

Menstrual cups are known, and various such cups are disclosed in U.S. Pat. No. 1,891,761 (Goddard), U.S. Pat. No. 1,996,242 (Hagedorn), U.S. Pat. No. 2,089,113 (Chalmers), Des. U.S. Pat. No. 323,212 (Crawford), and U.S. Pat. No. 5,827,248 (Crawford).

However, menstrual cups currently on the market are not suitable for use by all women. For example, standard menstrual cups are not suitable for use in women with a low cervix, prolapsed uterus or weakened pelvic floor. Standard menstrual cups can interfere with the cervix under these conditions, causing discomfort. In addition, a lower cervix can get stuck in or suctioned by a standard menstrual cup, resulting in difficulties removing the cup.

In addition, the use of a menstrual cup can result in leakage during use in certain positions, and/or spillage on removal.

Further, use of a menstrual cup restricts the opportunity for intercourse.

An alternative to a menstrual cup is the menstrual disc which is designed for placement in the vaginal fornix as opposed to the vaginal canal. The menstrual disc overcomes some disadvantages of the menstrual cup in that it is appropriate for use in women with a low cervix, and may be used during intercourse; however, the disc can be prone to mid-wear leaks, as well as challenging and messy to remove.

Therefore, there is a need for an improved menstrual cup or disc which overcomes one or more of the disadvantages of the prior art.

### SUMMARY OF THE INVENTION

An improved menstrual product has now been developed having a novel disc-like shape and enhanced features which prevent or minimize leakage and spills, and which is suitable for use during intercourse.

Thus, in one aspect, a menstrual disc is provided comprising:

i) a receptacle that has a posterior end, an anterior end and a perimeter, said receptacle comprising a wall having an inner surface and an outer surface, wherein the wall extends from an open top end to a closed bottom end, the inner surface of the wall defining a reservoir adapted to collect fluid, the wall of the reservoir extending from the bottom end to the anterior end having a slope that is less than the slope of the wall from the bottom end to the posterior end;

ii) a rim formed at the top end of the receptacle which extends around the perimeter of the receptacle; and optionally,

iii) a pull tab which extends from the anterior end of the receptacle; and/or

iv) a leak-resistant guard or shield extending from the rim at the anterior end to cover a portion of the open top end.

These and other aspects of the invention will become apparent by reference to the following drawings.

### BRIEF DESCRIPTION OF THE DRAWINGS

FIG. 1A is an isometric view of a menstrual disc in accordance with an embodiment of the invention;

FIG. 1B is a side view of the disc of FIG. 1A;

FIG. 1C is a top view of the disc of FIG. 1A;

FIG. 1D is a view of the anterior end of the disc of FIG. 1A;

FIG. 1E is a cross-sectional side view of the disc of FIG. 1A;

FIG. 2A is an isometric view of a menstrual disc in accordance with another embodiment of the invention;

FIG. 2B is a top view of the disc of FIG. 2A;

FIG. 2C is a side view of the disc in which its anterior end is downwardly tipped;

FIG. 3A illustrates a disc in accordance with the invention in position in the vagina; and

FIG. 3B illustrates the position of the disc when being inserted or removed from the vagina.

### DETAILED DESCRIPTION OF THE INVENTION

A menstrual disc is provided comprising a receptacle that has a posterior end, an anterior end and a perimeter, said receptacle comprising a wall having an inner surface and an outer surface, wherein the wall extends from an open top end to a closed bottom end, the inner surface of the wall defining a reservoir or cavity adapted to collect menstrual fluid, the wall of the reservoir extending from the bottom end to the anterior end having a positive slope that is less than the slope of the wall from the bottom end to the posterior end. The receptacle includes a rim that extends around the perimeter of the top end of the receptacle. The disc optionally includes a pull tab which is connected to the disc at the anterior end of the receptacle; and/or a leak-resistant guard extending from the rim at the anterior end to cover a portion of the open top end.

As will be appreciated by one of skill in the art, the menstrual disc is made of a biocompatible material, which is flexible, resilient, hypoallergenic and suitable for re-use, i.e. can be sanitized, for example, by boiling. Thus, the present menstrual disc may be made of rubber, silicone or a suitable thermoplastic elastomer, for example, an elastomer comprising a polyolefin polymer, semi-crystalline thermoplastic material or an amorphous elastomer such as polypropylene or a copolymer of ethylene and propylene (ethylene-propylene rubber (EPR)). As one of skill in the art will appreciate, the menstrual disc may be made of a combination of materials, e.g. the rim of the disc may comprise a stiffer material such as rubber, while the receptacle walls are made of a more pliable thermoplastic elastomer. Preferably the selected material(s) is medical grade.

The receptacle of the present menstrual disc generally comprises a shape that sits comfortably at the top of the vagina, e.g. in the vaginal fornix, and which fits around the cervix, without interfering with the cervix. The receptacle comprises a reservoir adapted to collect menstrual fluid and shaped to permit the cervix to sit within it without getting stuck or suctioned. The posterior end of the receptacle sits behind the cervix, while the anterior end of the receptacle sits in front of the cervix and thus, the disc fully surrounds the cervix. In embodiments, the receptable is circular or oval in cross-section. As one of skill in the art will appreciate, the disc may assume another shape, e.g. oblong, hour-glass or eight-shaped, which may be irregular, e.g. egg-shaped, but is appropriate for placement within the vaginal fornix, e.g. having curved edges.

In one embodiment, the disc is anti-rotational, having a shape which functions to maintain the disc in position during use covering the cervix while also minimizing or preventing rotation of the disc once in position. In particular, a disc having a length from the posterior end of the receptacle to its anterior end that is greater than the width of the receptacle (e.g. perpendicular to the receptacle length), the distance between the rim on a side of the receptacle to the rim on the opposing side of the receptacle. Such a disc may be oval in shape (e.g. having an oval cross-sectional shape in which the distance from the posterior to anterior end of the disc (the length) is longer than the distance from rim to rim of the disc perpendicular to the length (the width)). In one embodiment, the length of the receptacle may be in the range of about 50-100 mm, and the width may be in the range of about 40-90 mm.

The reservoir generally comprises a unique rounded or dome-like shape in which the slope of the reservoir wall extending from the bottom end to the anterior end of the disc is less than the slope of the reservoir wall extending from the bottom end to the posterior end of the disc, i.e. the slope of the reservoir wall is less steep from the bottom end to the anterior end, than the slope of the reservoir wall extending from the bottom end to the posterior end of the disc. This design feature of the disc concentrates the collected fluid in the deeper part of the reservoir at the posterior end of the disc which helps to minimize leaking or spillage from the shallower anterior end of the disc during use. In one embodiment, the depth of the reservoir is about 28-32 mm from the uppermost part of the rim at the open top end of the disc to the deepest part of the reservoir at the closed bottom end (e.g. within the posterior portion of the reservoir).

The reservoir of the disc is of an appropriate size to collect menstrual fluid. In this regard, menstrual discs with different reservoir sizes and fluid-holding capacities may be provided to accommodate different menstrual flows, including the spectrum from light to heavy menstrual flows. In this regard, the flow capacity of the disc may range from 30-50 mL, but may also be sized to accommodate flow volumes outside of this range. In one embodiment, due to its unique shape, the reservoir is sized to collect a larger volume of menstrual flow, e.g. about 40-50 mL, preferably about 42, 43, 44, 45, 46, 47 or 48 mL.

The receptacle includes a rim that extends around the perimeter of the top end of the receptacle. The length of the rim around the perimeter may vary from about 50-100 mm, depending on the size of the disc. The rim is designed to maintain the shape of the disc when in use, i.e. in position in the vagina, while being flexible enough to permit flattening of the rim to reduce its size during insertion. Accordingly, the rim comprises an increased thickness in comparison to the thickness of the wall of the disc. The thickness of the rim may vary across its width. The thickness of the rim may also vary with the material used to make the rim, which may be the same material used to make the rest of the disc or may be a different biocompatible material. For example, the rim may be one thickness when the menstrual disc is made of a stiffer material, such as a rubber, and may have an increased thickness when the menstrual disc is made of a softer material, e.g. a thermoplastic elastomer. In one embodiment, the thickness of the rim ranges from 1-6 mm, while the thickness of the wall of the disc may range from 0.15-1.0 mm. In an embodiment, the disc comprises a rim having a width from the open top end to the reservoir wall of about 8-14 mm, such as 10-12 mm.

The disc may include a leak-resistant guard or shield extending from the rim of the disc to cover a portion of the open top at the anterior end of the disc so as to prevent or minimize spilling of fluid from the disc at the anterior end. The leak-resistant guard is preferably an integral part of the rim, but will generally have a different thickness to that of the rim. In one embodiment, the leak-resistant guard comprises a thickness that is similar to or greater than the reservoir wall. The leak-resistant guard reduces or prevents spillage of the contents of the disc during removal of the disc in which the anterior end of the disc is tilted downwardly to facilitate its removal from the body. On tilting of the disc, any fluid in the disc will flow towards the edge of the reservoir at the anterior end. The guard functions to retain the fluid within the reservoir minimizing or preventing overflow into the surrounding area. The leak-resistant guard also functions to reduce or prevent accidental leakage of menstrual fluid from the disc that may occur when a user sits or squats and the position of the disc within the vagina tilts towards its anterior end. In this case, fluid in the disc may also flow towards the edge of the reservoir at the anterior end, but will be retained within the disc reservoir by the guard. The leak-resistant guard extends outwardly from the rim along the perimeter at the anterior end of the disc towards the posterior end of the disc to cover a portion of the reservoir at the anterior end. The guard extends a length from the rim that enables it to prevent or minimize leakage from the reservoir as described above, while not blocking collection of menstrual fluid. For example, the guard may extend about 15-40 mm from the rim, such as 20-30 mm from the rim to partially cover the reservoir. In one embodiment, the guard extends up to about 40% of the length of the disc.

The disc may also include a pull tab at its anterior end to facilitate removal of the disc from the body. The pull tab may take any appropriate form. For example, in one embodiment, the pull tab may extend from the rim at one side of the anterior end of the disc to the rim at the other side of the anterior end of the disc. Alternatively, the pull tab may be in the form of a ring, strip or other suitable configuration that extends from a central position at the anterior end of the disc that can readily be grasped to aid in removal of the disc.

The pull tab may be integrally formed with the disc or may be removable. The pull tab may be made of the same material as the disc or a different biocompatible material such as the materials described herein. The pull tab may comprise raised portions formed therein which function to aid a user to grip the pull tab during removal of the disc from the vagina.

The present disc may also include one or more volume markings on the inner wall surface of the receptacle to indicate the volume of fluid collected in the receptacle.

The present menstrual disc may be formed using well-established techniques such as techniques utilizing injection molding.

### DESCRIPTION OF AN EMBODIMENT OF THE INVENTION

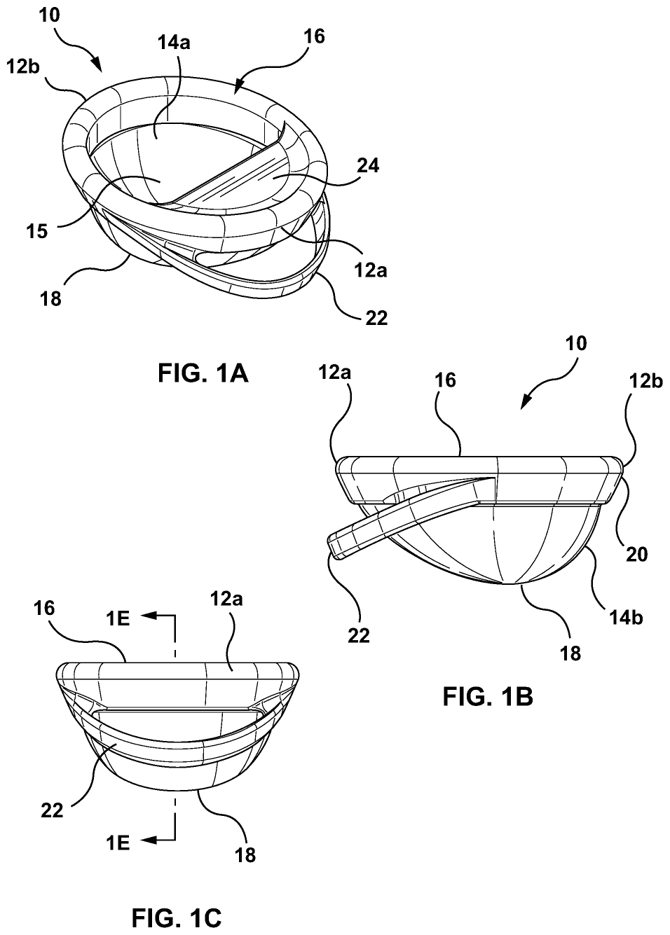

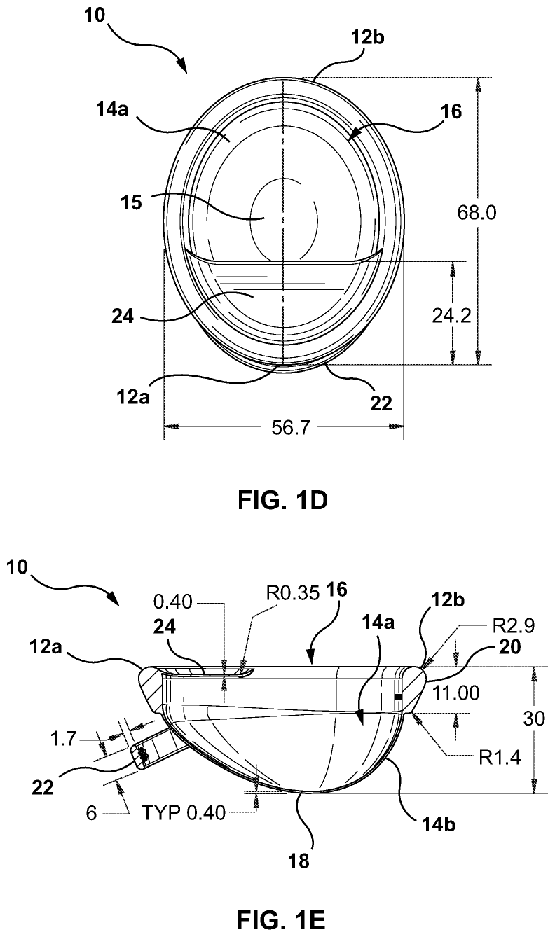

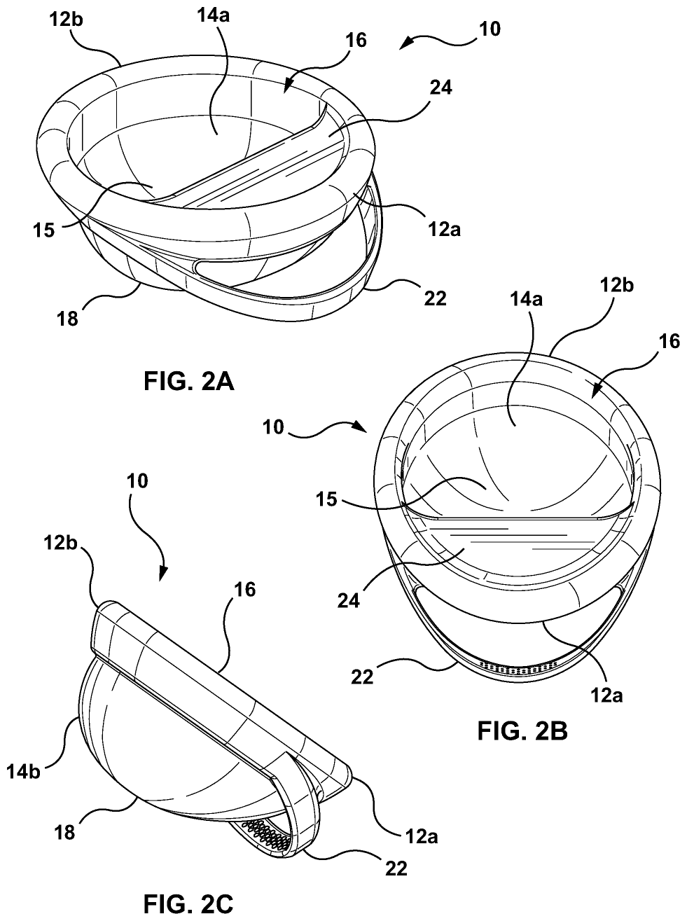

Reference is made to the Figures which illustrate embodiments of a menstrual disc in accordance with an embodiment of the invention. FIG. 1A/B illustrate a menstrual disc 10 including a receptacle having an anterior end 12a and a posterior end 12b. The receptacle comprises a wall with an inner wall surface 14a which defines a cavity or reservoir 15 adapted to collect fluid, and an opposed outer wall surface 14b. The receptacle extends from an open top end 16 to a closed bottom end 18.

The wall of the receptacle includes an upper thickened rim portion 20 which strengthens the top end 16 to maintain the disc in a pre-selected position within the vagina.

The thickness of the disc wall, i.e. the distance between the inner wall surface 12a and the outer wall surface 12b, is generally consistent, except within the upper rim portion 20. Various wall thicknesses for the rim portion and the main wall portion will be appropriate, as one of skill in the art will appreciate. The wall thickness of the main wall portion may also vary, being thicker in some areas, e.g. in the region extending directly from the rim portion 20 and thinner at the bottom of the receptacle.

The disc includes a pull tab 22 which extends from the rim portion 20 at one side of the anterior end 12a to the rim portion 20 at the other side of the anterior end 12a of the receptacle.

The disc also includes a leak-proof guard 24 at the anterior end 12a of the receptacle which extends from the rim 20 towards the posterior end 12b of the receptacle, and covers a portion of the open top end 16 of the receptacle.

In an embodiment shown (see FIG. 1C/E), the disc has an oval shape with the following dimensions: a length across the top end of about 68 mm, a width of about 56.7 mm at the central point and perpendicular to the length and a depth of about 30 mm from the rim 20 at the top end 16 to the deepest part of the reservoir at the bottom end 18. The leak-proof guard 24 extends about 24.2 mm from rim 20 along the length of the disc at the anterior end 12a to cover a portion of the open end 16. The wall of the reservoir is about 0.4 mm thick and the rim portion 20 decreases in thickness from about 2.9 mm thick at the top end to about 1.4 mm thick and has a width of about 11 mm. The pull tab 22 is about 1.7 mm thick and has a width of about 6 mm. As one of skill in the art will appreciate, these values may be varied without impact on the function of the disc. For example, the term “about” indicates that these values may be varied, for example, by about 10% from the recited value, greater or less, or by an amount that would not impact the function of the disc. A disc with this range of sizes has been determined to be suitable for use by most women, folds to the size of a tampon for easy insertion, holds at least 7 tampons of menstrual fluid, and to be safe for up to 12 hours of continuous wear. In addition, the oval shape minimizes or prevents rotation of the disc once in position such that the pull tab remains accessible and the leak-proof guard remains is in an effective position to minimize or prevent spillage from the disc.

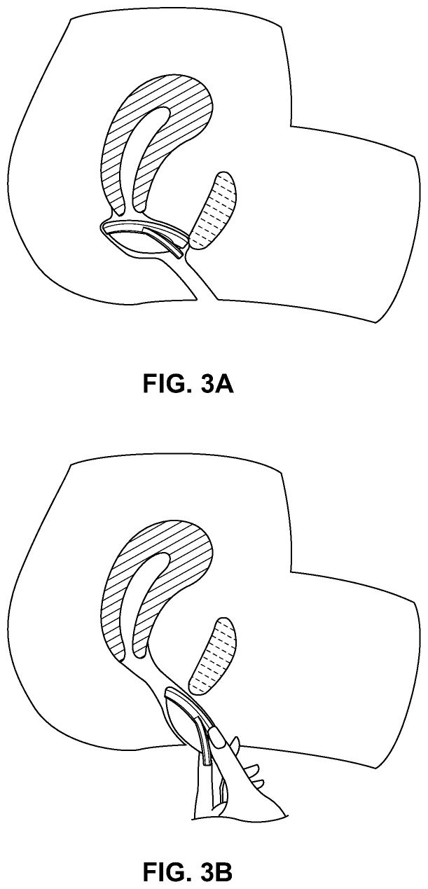

In use, the menstrual disc may be prepared for insertion into the vagina, e.g. sterilizing or washing as necessary. The receptacle of the menstrual disc may be pinched or squeezed on either side of the rim between the anterior and posterior ends of the disc, or along its width if the width is less than the length of the receptacle. The rim may or may not include finger-receiving indents to aid in gripping of the rim to pinch/squeeze the disc. The pinching together of the sides of the receptacle results in a flattening of the disc (opposing edges of the rim portion 20 are touching) along its length. The disc can then be inserted like a tampon into the vagina, posterior end 12b first. The disc is positioned in the vaginal fornix, with the posterior end 12b behind the cervix and the anterior end 12a in front of the cervix such that the disc surrounds the cervix as shown in FIG. 3A and the reservoir 15 collects fluid expelled from the cervix.

To remove the menstrual disc, the user grasps the rim of the disc its anterior end 12a, or pull tab 22 if present, and gently pulls the disc out of the vagina. When the disc is close to the entrance of the vagina, the rim can be pinched to remove the disc the rest of the way as shown in FIG. 3B. It is clear from FIG. 3B that the disc is tilted downwardly towards its anterior end 12a on removal and that any fluid within the disc will accumulate at the anterior end 12a of the disc. Thus, a disc incorporating a leak-proof guard 24 as described herein will minimize or prevent spilling of fluid from within the reservoir 15 of the disc.

It will be appreciated by those skilled in the art that the invention can take many forms, and that such forms are within the scope of the invention as claimed. Therefore, the spirit and scope of the appended claims should not be limited to the descriptions of the preferred versions contained herein.

1. A menstrual disc comprising:
i) a receptacle that has a posterior end, an anterior end and a perimeter, said receptacle comprising a wall having an inner surface and an outer surface, wherein the wall extends from an open top end to a closed bottom end, the inner surface of the wall defining a reservoir adapted to collect fluid, wherein the wall of the reservoir extending from the bottom end to the anterior end has a slope that is less than the slope of the wall from the bottom end to the posterior end; and
ii) a rim formed at the top end of the receptacle which extends around the perimeter of the receptacle,
wherein the disc is made of a biocompatible, flexible material.

2. The menstrual disc of claim 1, comprising a pull tab at the anterior end of the disc.

3. The menstrual disc of claim 2, wherein the pull tab extends from the rim at one side of the anterior end of the disc to the rim at the other side of the anterior end of the receptacle.

4. The menstrual disc of claim 2, wherein the pull tab is a ring at the anterior end of the disc.

5. The menstrual disc of claim 2, wherein the pull tab is a strip at the anterior end of the disc.

6. The menstrual disc of claim 2, wherein the pull tab is removable.

7. The menstrual disc of claim 1, comprising a leak-resistant guard extending from the rim at the anterior end of the disc to cover a portion of the open top end to minimize or prevent spilling of fluid from the anterior end of the disc.

8. The menstrual disc of claim 7, wherein the leak-resistant guard comprises a thickness which is equal to or greater than the thickness of the receptacle wall.

9. The menstrual disc of claim 7, wherein the leak-resistant guard extends up to about 40% of a length of the disc.

10. The menstrual disc of claim 1, wherein the disc comprises a circular cross-sectional shape.

11. The menstrual disc of claim 1, wherein the disc comprises an oval cross-sectional shape.

12. The menstrual disc of claim 1, having a length from the posterior end of the receptacle to its anterior end which is greater than the distance perpendicular to the receptacle length between the rim on a side of the receptacle to a rim on an opposing side of the receptacle.

13. The menstrual disc of claim 1, which is made of rubber, silicone, a thermoplastic elastomer or a combination thereof.

14. The menstrual disc of claim 1, wherein the reservoir has a depth at the posterior end of the disc that is greater than the depth of the reservoir as the anterior end of the disc.

15. The menstrual disc of claim 1, wherein the rim is thicker than the wall of the receptacle.
